(19) United States
(12) Patent Application Publication
(10) Pub. No.: US 2006/0069135 A1
(43) Pub. Date: Mar. 30, 2006

(54) Benzimidazole derivatives and their use for modulating the gaba-a receptor complex

(76) Inventors: Lene Teuber, Varlose (DK); Janus S Larsen, Holbak (DK)

Correspondence Address: BIRCH STEWART KOLASCH & BIRCH, PO BOX 747, FALLS CHURCH, VA, 22040-0747, US

(21) Appl. No.: 10/546,437

(22) PCT Filed: Apr. 1, 2004
(86) PCT No.: PCT/EP04/50417

§ 371 Date: Aug. 19, 2005

Related U.S. Application Data
(60) Provisional application No. 60/459,980, filed on Apr. 4, 2003.

(30) Foreign Application Priority Data
Apr. 3, 2003 (DK) PA 2003 00509

(51) Int. Cl. A61K 31/426; A61K 31/42; A61K 31/4184; C07D 417/02; C07D 413/02; C07D 403/02

(52) U.S. Cl. 514/365; 514/394; 548/304.7; 548/304.4; 548/181; 548/240; 514/372; 514/378

(57) ABSTRACT

This invention relates to novel benzimidazole derivatives, pharmaceutical compositions containing these compounds, and methods of treatment therewith.

The compounds of the invention are useful in the treatment of central nervous system diseases and disorders, which are responsive to modulation of the GABA_{A} receptor complex, and in particular for combating anxiety and related diseases.

### TECHNICAL FIELD

This invention relates to novel benzimidazole derivatives, pharmaceutical compositions containing these compounds, and methods of treatment therewith.

The compounds of the invention are useful in the treatment of central nervous system diseases and disorders, which are responsive to modulation of the GABA_{A} receptor complex, and in particular for combating anxiety and related diseases.

### BACKGROUND ART

The modulatory sites on the GABA_{A} receptor complex, such as for example the benzodiazepine receptor, are the target for anxiolytic drugs, such as the classical anxiolytic benzodiazepines.

Multiple isoforms of the GABA_{A} receptor exist; each receptor is a pentameric complex comprising subunits drawn from α₁₋₆, β₁₋₃, γ₁₋₃, δ, ε, and θ subunit isoforms. The classical anxiolytic benzodiazepines show no subtype selectivity. It has been suggested that one of the key elements in the disadvantages of the classical benzodiazepanes (such as sedation, dependency, and cognitive impairment) is relates to the α1 subunit of the GABA_{A} receptors. Thus compounds with selectivity for the α2 and/or α3 subunits over the α1 subunit are expected to have an improved side effect profile.

EP 616807 describes benzimidazole compounds for use as benzodiazepine receptor ligands.

WO 96/33194, WO 96/33191 and WO 96/33192 describe benzimidazole compounds having affinity for the GABA receptor complex.

WO 98/34923 describes phenylbenzimidazole derivatives as ligands for the GABA receptor complex.

WO 98/17651 and WO 00/78728 describe benzimidazole compounds for use as e.g. anaesthetics.

However, there is a continued strong need to find compounds with an optimized pharmacological profile. Furthermore, there is a strong need to find effective compounds without unwanted side effects associated with older compounds.

### SUMMARY OF THE INVENTION

In its first aspect, the invention provides a compound of the Formula I: 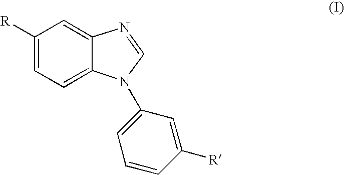
or an N-oxide thereof, or any of its isomers or any mixture of its isomers, or a pharmaceutically acceptable salt thereof,
wherein R and R′ are defined as below.

In its second aspect, the invention provides a pharmaceutical composition, comprising a therapeutically effective amount of a compound of the invention, or an N-oxide thereof, or any of its isomers or any mixture of its isomers, or a pharmaceutically acceptable salt thereof, together with at least one pharmaceutically acceptable carrier, excipient or diluent.

In a further aspect, the invention provides the use of a compound of the invention, or an N-oxide thereof, or any of its isomers or any mixture of its isomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to modulation of the GABA_{A} receptor complex in the central nervous system.

In a still further aspect, the invention relates to a method for treatment, prevention or alleviation of a disease or a disorder or a condition of a living animal body, including a human, which disorder, disease or condition is responsive to modulation of the GABA_{A} receptor complex in the central nervous system, which method comprises the step of administering to such a living animal body in need thereof a therapeutically effective amount of a compound of the invention, or an N-oxide thereof, or any of its isomers or any mixture of its isomers, or a pharmaceutically acceptable salt thereof.

Other objects of the invention will be apparent to the person skilled in the art from the following detailed description and examples.

### DETAILED DISCLOSURE OF THE INVENTION

Substituted Benzimidazole Derivatives

In its first aspect the present invention provides a compound of general formula (I): 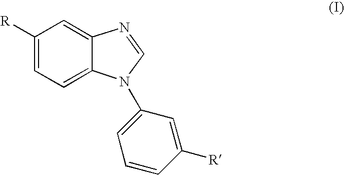
or an N-oxide thereof, or any of its isomers or any mixture of its isomers,
or a pharmaceutically acceptable salt thereof,
wherein
R represents hydroxyalkyl, alkoxyalkyl, cycloalkoxyalkyl, cycloalkylalkoxyalkyl, alkenyloxyalkyl, or alkynyloxyalkyl; and
R′ represents a 5-membered heterocyclic ring;
   which heterocyclic ring may optionally be substituted with one or more substituents independently selected from the group consisting of halo, hydroxy, amino, alkylamino, aminoalkyl, cyano, nitro, trifluoromethyl, trifluoromethoxy, alkoxy, cycloalkoxy, alkyl, cycloalkyl, cycloalkylalkyl, alkenyl, and alkynyl.

In one embodiment, R represents hydroxymethyl, alkoxymethyl, or cycloalkylalkoxymethyl. In a further embodiment, R represents 1-hydroxyethyl, 1-alkoxyethyl, 1-(cycloalkylalkoxy)ethyl, 1-(alkenyloxy)ethyl, or 1-(alkynyloxy)ethyl. In a special embodiment, R represents alkoxyalkyl. In a yet further embodiment, R represents 2-hydro-2-propyl, 2-alkoxy-2-propyl, or 2-(cycloalkylalkoxy)-2-propyl.

In a still further embodiment, R represents alkoxyalkyl. In a special embodiment, R represents alkoxymethyl, such as methoxymethyl, ethoxymethyl, isopropoxymethyl, or propoxymethyl. In a further embodiment, R represents alkoxyethyl, in particular 1-alkoxy-ethyl, such as 1-methoxyethyl, 1-ethoxyethyl, 1-propoxyethyl, 1-isopropoxyethyl. In a still further embodiment, R represents alkoxypropyl, in particular 2-alkoxy-2-propyl, such as 2-methoxy-2-propyl, 2-ethoxy-2-propyl, 2-propoxy-2-propyl and 2-isopropoxy-2-propyl.

In a further embodiment, R represents cycloalkylalkoxyalkyl. In a special embodiment, R represents cycloalkylalkoxymethyl, such as cyclopropylmethoxymethyl. In a further embodiment R represents cycloalkylalkoxyethyl, in particular 1-(cycloalkylalkoxy)ethyl, such as 1-(cyclopropylmethoxy)ethyl. In a still further embodiment, R represents cycloalkylalkoxypropyl, in particular 2-(cycloalkylalkoxy)-2-propyl, such as 2-(cyclopropylmethoxy)-2-propyl.

In a still further embodiment, R represents hydroxyalkyl. In a special embodiment, R represents hydroxymethyl. In a further embodiment, R represents hydroethyl, such as 1-hydroxyethyl. In a still further embodiment, R represents hydroxypropyl, such as 2-hydroxy-2-propyl.

In a further embodiment, R represents alkenyloxyalkyl. In one embodiment, R represent alkenyloxyethyl, in particular 1-(alkenyloxy)ethyl such as 1-(allyloxy)ethyl.

In a further embodiment R represents alkynyloxyalkyl. In one embodiment, R represents alkynyloxyethyl, in particular 1-(alkynyloxy)ethyl, such as 1-(propargyloxy)ethyl.

In a further embodiment, R′ represents imidazolyl, pyrazolyl, thienyl, furyl, isoxazolyl, thiazolyl, pyrrolyl, or pyrrolidinyl. In a still further embodiment, R′ represents imidazolyl, pyrazolyl, thienyl, or furyl. In a special embodiment, R′ represents imidazolyl, such as 1-imidazolyl, In a further embodiment, R′ represents pyrazolyl, such as 1-pyrazolyl. In a still further embodiment, R′ represents thienyl, such as 2-thienyl. In a further embodiment, R′ represents furyl, such as 3-furyl. In a still further embodiment, R′ represents isoxazolyl, such as 3-isoxazolyl. In a further embodiment, R′ represents pyrrolidinyl, such as 1-pyrrolidinyl. In a still further embodiment, R′ represents thiazolyl, such as 2-thiazolyl. In a further embodiment, R′ represents pyrrolyl, such as 1-pyrrolyl.

In a special embodiment the chemical compound of the invention is
1-(3-(1-Imidazolyl)phenyl)-5-(methoxymethyl)benzimidazole;
1-(3-(1-Imidazolyl)phenyl)-5-(ethoxymethyl)benzimidazole;
1-(3-(1-Imidazolyl)phenyl)-5-(isopropoxymethyl)benzimidazole;
1-(3-(1-Imidazolyl)phenyl)-5-(propoxymethyl)benzimidazole;
1-(3-(1-Imidazolyl)phenyl)-5-(cyclopropylmethoxymethyl)benzimidazole;
1-(3-(1-Pyrazolyl)phenyl)-5-(methoxymethyl)benzimidazole;
1-(3-(1-Pyrazolyl)phenyl)-5-(hydroxymethyl)benzimidazole;
1-(3-(1-Pyrazolyl)phenyl)-5-(ethoxymethyl)benzimidazole;
1-(3-(1-Pyrazolyl)phenyl)-5-(isopropoxymethyl)benzimidazole;
1-(3-(1-Pyrazolyl)phenyl)-5-(propoxymethyl)benzimidazole;
1-(3-(1-Pyrazolyl)phenyl)-5-(cyclopropylmethoxymethyl)benzimidazole;
1-(3-(3-Furyl)phenyl)-5-(methoxymethyl)benzimidazole;
1-(3-(3-Furyl)phenyl)-5-(hydroxymethyl)benzimidazole;
1-(3-(3-Furyl)phenyl)-5-(ethoxymethyl)benzimidazole;
1-(3-(3-Furyl)phenyl)-5-(isopropoxymethyl)benzimidazole;
1-(3-(3-Furyl)phenyl)-5-(propoxymethyl)benzimidazole;
1-(3-(3-Furyl)phenyl)-5-(cyclopropylmethoxymethyl)benzimidazole;
1-(3-(2-Thienyl)phenyl)-5-(methoxymethyl)benzimidazole;
1-(3-(2-Thienyl)phenyl)-5-(hydroxymethyl)benzimidazole;
1-(3-(2-Thienyl)phenyl)-5-(ethoxymethyl)benzimidazole;
1-(3-(2-Thienyl)phenyl)-5-(isopropoxymethyl)benzimidazole;
1-(3-(2-Thienyl)phenyl)-5-(propoxymethyl)benzimidazole;
1-(3-(2-Thienyl)phenyl)-5-(cyclopropylmethoxymethyl)benzimidazole;
5-(1-Hydroxyethyl)-1-(3-(3-isoxazolyl)phenyl)benzimidazole;
5-(1-(Alloxy)ethyl)-1-(3-(3-isoxazolyl)phenyl)benzimidazole;
5-(1-Propargyloxy)ethyl)-1-(3-(3-isoxazolyl)phenyl)benzimidazole;
5-(1-Methoxyethyl)-1-(3-(3-isoxazolyl)phenyl)benzimidazole;
5-(1-Ethoxyethyl)-1-(3-(3-isoxazolyl)phenyl)benzimidazole;
5-(1-Propoxyethyl)-1-(3-(3-isoxazolyl)phenyl)benzimidazole;
5-(1-Isopropoxyethyl)yl)-(3-(3-isoxazolyl)phenyl)benzimidazole;
5-(1-(Cyclopropylmethoxy)ethyl)-1-(3-(3-isoxazolyl)phenyl)benzimidazole;
5-(1-Hydroxyethyl)-1-(3-(1-imidazolyl)phenyl)benzimidazole;
5-(1-Methoxyethyl)-1-(3-(1-imidazolyl)phenyl)benzimidazole;
5-(1-Ethoxyethyl)-1-(3-(1-imidazolyl)phenyl)benzimidazole;
5-(1-Propoxyethyl)-1-(3-(1-imidazolyl)phenyl)benzimidazole;
5-(1-Isopropoxyethyl)yl)-1-(3-(1-imidazolyl)phenyl)benzimidazole;
5-(1-(Cyclopropylmethoxy)ethyl)-1-(3-(1-imidazolyl)phenyl)benzimidazole;
5-(1-Hydroxyethyl)-1-(3-(1-pyrrolidinyl)phenyl)benzimidazole;
5-(1-Methoxyethyl)-1-(3-(1-pyrrolidinyl)phenyl)benzimidazole;
5-(1-Ethoxyethyl)-1-(3-(1-pyrrolidinyl)phenyl)benzimidazole;
5-(1-Propoxyethyl)-1-(3-(1-pyrrolidinyl)phenyl)benzimidazole;
5-(1-Isopropoxyethyl)-1-(3-(1-pyrrolidinyl)phenyl)benzimidazole;
5-(2-(Cyclopropylmethoxy)ethyl)-1-(3-(1-pyrrolidinyl)phenyl)benzimidazole;
5-(2-Hydroxy-2-propyl)-1-(3-(1-pyrrolidinyl)phenyl)benzimidazole;
5-(2-Methoxy-2-propyl)-1-(3-(1-pyrrolidinyl)phenyl)benzimidazole;
5-(2-Ethoxy-2-propyl)-1-(3-(1-pyrrolidinyl)phenyl)benzimidazole;
5-(2-Propoxy-2-propyl)-1-(3-(1-pyrrolidinyl)phenyl)benzimidazole;
5-(2-Isopropoxy-2-propyl)-1-(3-(1-pyrrolidinyl)phenyl)benzimidazole;
5-(2-(Cyclopropylmethoxy)-2-propyl)-1-(3-(1-pyrrolidinyl)phenyl)benzimidazole;
5-(Hydroxymethyl)-1-(3-(2-thiazolyl)phenyl)benzimidazole;
5-(Hydroxymethyl)-1-(3-(3-imidazolyl)phenyl)benzimidazole;
5-(Hydroxymethyl)-1-(3-(3-isoxazolyl)phenyl)benzimidazole;
5-(Hydroxymethyl)-1-(3-(2-pyrrolyl)phenyl)benzimidazole;
5-(Methoxymethyl)-1-(3-(2-thiazolyl)phenyl)benzimidazole;
5-(Methoxymethyl)-1-(3-(3-isoxazolyl)phenyl)benzimidazole;
5-(Ethoxymethyl)-1-(3-(3-isoxazolyl)phenyl)benzimidazole;
5-(Isopropoxymethyl)-1-(3-(3-isoxazolyl)phenyl)benzimidazole;
5-(Propoxymethyl)-1-(3-(3-isoxazolyl)phenyl)benzimidazole;
5-(Cyclopropylmethoxymethyl)-1)-(3-(3-isoxazolyl)phenyl)benzimidazole;
5-(Methoxymethyl)-1-(3-(1-pyrrolyl)phenyl)benzimidazole;
5-(Ethoxymethyl)-1-(3-(1-pyrrolyl)phenyl)benzimidazole;
5-(Isopropoxymethyl)-1-(3-(1-pyrrolyl)phenyl)benzimidazole;
5-(Propoxymethyl)-1-(3-(1-pyrrolyl)phenyl)benzimidazole;
5-(Cyclopropylmethoxymethyl)-1-(3-(1-pyrrolyl)phenyl)benzimidazole;
5-(Ethoxymethyl)-1-(3-(2-thiazolyl)phenyl)benzimidazole;
5-(Isopropoxymethyl)-1-(3-(2-thiazolyl)phenyl)benzimidazole;
5-(Propoxymethyl)-1-(3-(2-thiazolyl)phenyl)benzimidazole;
5-(Cyclopropylmethoxymethyl)-1-(3-(2-thiazolyl)phenyl)benzimidazole;
or an N-oxide thereof, or any of its isomers or any mixture of its isomers,
or a pharmaceutically acceptable salt thereof.

Any combination of two or more of the embodiments as described above is considered within the scope of the present invention.

Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkenyl group designates a carbon chain containing one or more double bonds, including di-enes, tri-enes and poly-enes. In a preferred embodiment the alkenyl group of the invention comprises of from two to six carbon atoms (C₂₋₆-alkenyl), including at least one double bond. In a most preferred embodiment the alkenyl group of the invention is ethenyl; 1- or 2-propenyl; 1-, 2- or 3-butenyl, or 1,3-butdienyl; 1-, 2-, 3-, 4- or 5-hexenyl, or 1,3-hexdienyl, or 1,3,5-hextrienyl.

In the context of this invention an alkynyl group designates a carbon chain containing one or more triple bonds, including di-ynes, tri-ynes and poly-ynes. In a preferred embodiment the alkynyl group of the invention comprises of from two to six carbon atoms (C₂₋₆-alkynyl), including at least one triple bond. In its most preferred embodiment the alkynyl group of the invention is ethynyl; 1-, or 2-propynyl; 1-, 2-, or 3-butynyl, or 1,3-butdiynyl; 1-, 2-, 3-, 4-pentynyl, or 1,3-pentdiynyl; 1-, 2-, 3-, 4-, or 5-henynyl, or 1,3-hexdiynyl or 1,3,5-hextriynyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

Alkoxy means O-alkyl, wherein alkyl is as defined above.

Alkoxyalkyl means alkoxy as above and alkyl as above, meaning for example, methoxymethyl.

Cycloalkoxy means O-cycloalkyl, wherein cycloalkyl is as defined above.

Cycloalkylalkyl means cycloalkyl as above and alkyl as above, meaning for example, cyclopropylmethyl.

In the context of this invention alkylamino designates —NH-alkyl or —N-(alkyl)₂, wherein alkyl is as defined above.

In the context of this invention a 5-membered heterocyclic ring designates a 5-membered monocyclic group, and which group holds one or more heteroatoms in its ring structure. Preferred heteroatoms include nitrogen (N), oxygen (O), and sulphur (S). The ring structure may in particular be aromatic (i.e. a heteroaryl), saturated or partially saturated.

Examples of preferred aromatic heterocyclic monocyclic 5-membered groups of the invention include furan, in particular 2- or 3-furyl; thiophene, in particular 2- or 3-thienyl; pyrrole (azole), in particular 1-, 2- or 3-pyrrolyl; oxazole, in particular oxazol-(2-, 4- or 5-)yl; thiazole, in particular thiazol-(2-, 4-, or 5-)yl; imidazole, in particular imidazol-(1-, 2-, 4- or 5-)yl; pyrazole, in particular pyrazol-(1-, 3-, 4- or 5-)yl; isoxazole, in particular isoxazol-(3-, 4- or 5-)yl; isothiazole, in particular isothiazol-(3-, 4- or 5-)yl; 1,2,3-oxadiazole, in particular 1,2,3-oxadiazol-(4- or 5-)yl; 1,2,4-oxadiazole, in particular 1,2,4-oxadiazol-(3- or 5-)yl; 1,2,5-oxadiazole, in particular 1,2,5-oxadiazol-(3- or 4-)yl; 1,3,4-oxadiazole, in particular 1,3,4-oxadiazol-(2- or 5-)yl; 1,2,3-triazole, in particular 1,2,3-triazol-(1-, 4- or 5-)yl; 1,2,4-triazole, in particular 1,2,4-triazol(1-, 3- or 4-)yl; 1,2,4-thiadiazole, in particular 1,2,4-thiadiazol-(3- or 5-)yl; 1,2,5-thiadiazole, in particular 1,2,5-thiadiazol-(3- or 4-)yl; 1,3,4-thiadiazole, in particular 1,3,4-thiadiazol-(2- or 5-)yl; and tetrazole, in particular tetrazol-(1- or 5-)yl.

Examples of preferred saturated or partially saturated heterocyclic monocyclic 5-membered groups of the invention include 1,3-dioxolan, in particular 1,3-dioxolan-(2- or 4-)yl; imidazolidine, in particular imidazolidin-(1-,2-,3-,4- or 5-)yl; 2-imidazoline, in particular 2-imidazolin-(1-,2-,4- or 5-)yl; 3-imidazoline, in particular 3-imidazolin-(1-,2-,4- or 5-)yl; 4-imidazoline, in particular 4-imidazolin-(1-,2-,4- or 5-)yl; dihydro-oxazole (oxazoline), in particular dihydro-oxazol-(2-,4- or 5-)yl; tetrahydro-oxazole (oxazolidine), in particular tetrahydro-oxazol-(2-,4- or 5-)yl; 1,2,3-oxadiazoline, in particular 1,2,3-oxadiazol-(4- or 5-)yl; 1,2,4-oxadiazoline, in particular 1,2,4-oxadiazolin-(3- or 5-)yl; 1,2,5-oxadiazoline, in particular 1,2,5-oxadiazolin-(3- or 4-)yl; 1,2,3-oxadiazolidine, in particular 1,2,3-oxadiazolidin-(4- or 5-)yl; 1,2,4-oxadiazolidine, in particular 1,2,4-oxadiazolidin-(3- or 5-)yl; 1,2,5-oxadiazolidine, in particular 1,2,5-oxadiazolidin-(3- or 4-)yl; dihydro-pyrrole (pyrroline), in particular dihydro-pyrrol-(1-,2- or 3-)yl; tetrahydro-pyrrole (pyrrolidine), in particular tetrahydro-pyrrol-(1-,2- or 3-)yl; pyrazolidine, in particular pyrazolidin-(1-,2-,3-,4- or 5-)yl; 2-pyrazoline, in particular 2-pyrazolin-(1-,3-,4- or 5-)yl; and 3-pyrazoline, in particular 3-pyrazolin-(1-,3-,4- or 5-)yl.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art. Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention include alkali metal salts such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Steric Isomers

It will be appreciated by those skilled in the art that the compounds of the present invention may contain one or more chiral centres and that such compounds exist in the form of isomers.

The racemates of these isomers and the individual isomers themselves are within the scope of the present invention.

Methods for the resolvation of optical isomers, known to those skilled in the art may be used, and will be apparent to the average worker skilled in the art. Such methods include those discussed by J. Jaques, A. Collet, and S. Wilen in “Enantiomers, Racemates, and Resolutions”, John Wiley and Sons, New York (1981).

Optical active compounds can also be prepared from optical active starting materials.

N-oxides

In the context of this invention an N-oxide designates an oxide derivative of a nitrogen containing compound, e.g. N-containing heterocyclic compounds capable of forming such N-oxides, and compounds holding one or more amino groups. For example, the N-oxide of a compound containing a pyridyl may be the 1-oxy-pyridin-2, -3 or -4-yl derivative.

N-oxides of the compounds of the invention may be prepared by oxidation of the corresponding nitrogen base using a conventional oxidizing agent such as hydrogen peroxide in the presence of an acid such as acetic acid at an elevated temperature, or by reaction with a peracid such as peracetic acid in a suitable solvent, e.g. dichloromethane, ethyl acetate or methyl acetate, or in chloroform or dichloromethane with 3-chloroperoxybenzoic acid.

Labelled Compounds

The compounds of the invention may be used in their labelled or unlabelled form. In the context of this invention “label” stands for the binding of a marker to the compound of interest that will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

Biological Activity

Compounds of the invention are capable of modulating the GABA_{A} receptor complex. They may be tested for their ability to bind to the GABA_{A} receptor complex, including specific subunits thereof.

The compounds of the present invention, being ligands for the benzodiazepine binding site on GABA_{A} receptors, are therefore of use in the treatment and/or prevention of a variety of disorders of the central nervous system. Thus in further aspect, the compounds of the invention are considered useful for the treatment, prevention or alleviation of a disease, disorder or condition responsive to modulation of the GABA_{A} receptor complex in the central nervous system.

In a special embodiment, the compounds of the invention are considered useful for the treatment, prevention or alleviation of

anxiety disorders, such as panic disorder with or without agoraphobia, agoraphobia without history of panic disorder, animal and other phobias including social phobias, obsessive-compulsive disorder, and generalized or substance-induced anxiety disorder;

stress disorders including post-traumatic and acute stress disorder;

sleep disorders;

memory disorder;

neuroses;

convulsive disorders, for example epilepsy, or febrile convulsions in children;

migraine;

depressive or bipolar disorders, for example single-episode or recurrent major depressive disorder, dysthymic disorder, bipolar I and bipolar II manic disorders, and cyclothymic disorder,

psychotic disorders, including schizophrenia;

neurodegeneration arising from cerebral ischemia;

attention deficit hyperactivity disorder;

pain and nociception;

emesis, including acute, delayed and anticipatory emesis, in particular emesis induced by chemotherapy or radiation;

motion sickness, post-operative nausea and vomiting;

eating disorders including anorexia nervosa and bulimia nervosa;

premenstrual syndrome;

muscle spasm or spasticity, e.g. in paraplegic patients;

the effects of substance abuse or dependency, including alcohol withdrawal;

cognitive disorders, such as Alzheimer's disease; and

disorders of circadian rhythm, e.g. in subjects suffering from the effects of jet lag or shift work.

Preferably the compounds of the invention are considered useful for the treatment, prevention or alleviation of anxiety disorders, such as panic disorder with or without agoraphobia, agoraphobia without history of panic disorder, animal and other phobias including social phobias, obsessive-compulsive disorder, and generalized or substance-induced anxiety disorder;

Further, the compounds of the invention may be useful as radioligands in assays for detecting compounds capable of binding to the human GABA_{A} receptor.

Pharmaceutical Compositions

In another aspect the invention provides novel pharmaceutical compositions comprising a therapeutically effective amount of a compound of the invention.

While a compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising a compound of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### EXAMPLES

The invention is further illustrated with reference to the following examples, which are not intended to be in any way limiting to the scope of the invention as claimed.

### Examples

The invention is further illustrated with reference to the following examples, which are not intended to be in any way limiting to the scope of the invention as claimed.
Example 1 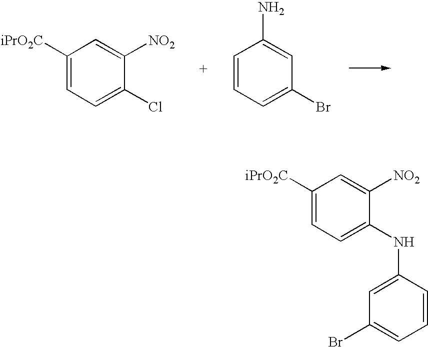
Isopropyl 4-(3-bromophenylamino)-3-nitrobenzoate. To a solution of isopropyl 4-chloro-3-nitrobenzoate (10.9 g; 45 mmol) in anhydrous N-methyl 2-pyrrolidinone (30 ml) was added 3-bromoaniline (7.3 ml; 67.5 mmol) and triethylamine (6.3 ml; 45 mmol). The mixture was stirred at 110° C. overnight. The cooled mixture was poured into ice-water (300 ml) and the precipitate was filtered off, washed with water and dried. 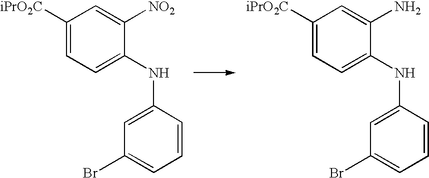
Isopropyl 3-amino-4-(3-bromophenylamino)benzoate. The above product was dissolved in a mixture of tetrahydrofurane (100 ml) and ethanol (50 ml) and was hydrogenated at ambient pressure using Raney Ni as the catalyst until the hydrogen uptake had ceased. The resulting mixture was filtered through filter aid and the filtrate was evaporated under reduced pressure. The residue was used for the next step. 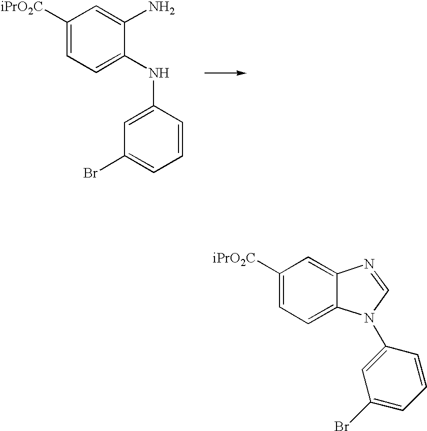
1-(3-Bromophenyl)-5-(isopropyloxycarbonyl)benzimidazole. To the above product was added formic acid (25 ml) and the mixture was heated to reflux for 1.5 hours. The cooled mixture was concentrated under reduced pressure, and the concentrate was partitioned between saturated, aqueous sodium carbonate and ethyl acetate. The organic phase was dried over magnesium sulfate and evaporated to dryness. The residue was eluted through silica gel using a mixture of ligroin and ethyl acetate as the eluent. Removal of solvent left the desired product (13.2 g; 82% three steps). 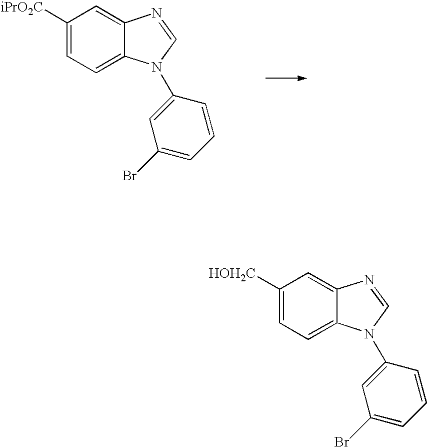
1-(3-Bromophenyl)-5-(hydroxymethyl)benzimidazole. To a solution of the above product (9.1 g; 25.3 mmol) in anhydrous diethyl ether (200 ml) was added lithium aluminum hydride (1.03 g; 27.1 mmol) in portions over 40 min. The resultant mixture was stirred at ambient temperature over night. Water (10 ml) was added drop-wise followed by addition of hydrochloric acid (20 ml; 4M) and additional water (200 ml). The phases were separated and the aqueous phase was rendered alkaline by addition of saturated, aqueous sodium carbonate. Extraction with ethyl acetate and column chromatographic work-up on silica gel using ethyl acetate as the eluent afforded the desired product as a yellow oil. Yield: 1.7 g (22%). 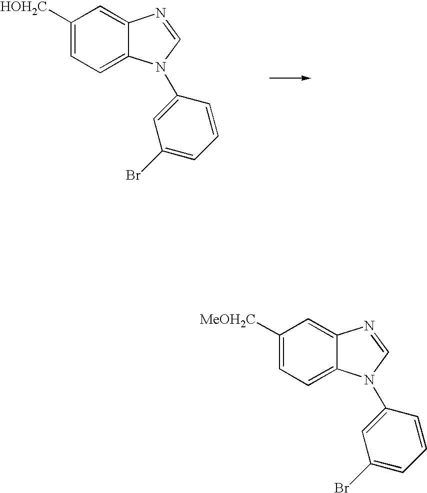
1-(3-Bromophenyl)-5-(methoxymethyl)benzimidazole. A solution of the above product (1.7 g, 5.6 mmol) in dimethyl formamide (15 ml) was cooled to 0° C. and sodium hydride (0.24 g 60% dispersion in mineral oil, 6 mmol) was added. When the evolution of hydrogen had ceased, iodomethane (0.43 ml, 7 mmol) was added and the mixture was stirred at ambient temperature for 1 hour. Hereafter, water (50 ml) was added and the mixture was extracted with ethyl acetate. The extract was washed with water, dried over magnesium sulfate and concentrated under reduced pressure. The concentrate was purified by elution through silica gel with a mixture of ethyl acetate and petroleum ether (1:1). Yield: 1 g (56%).
1-(3-Bromophenyl)-5-(ethoxymethyl)benzimidazole is prepared analogously from 1-(3-bromophenyl)-5-(hydroxymethyl)benzimidazole by alkylating with iodoethane.
1-(3-Bromophenyl)-5-(isopropoxymethyl)benzimidazole is prepared analogously from 1-(3-bromophenyl)-5-(hydroxymethyl)benzimidazole by alkylating with 2-bromopropane.
1-(3-Bromophenyl)-5-(propoxymethyl)benzimidazole is prepared analogously from 1-(3-bromophenyl)-5-(hydroxymethyl)benzimidazole by alkylating with 1-bromopropane.
1-(3-Bromophenyl)-5-(cyclopropylmethoxymethyl)benzimidazole is prepared analogously from 1-(3-bromophenyl)-5-(hydroxymethyl)benzimidazole by alkylating with (bromomethyl)cyclopropane. 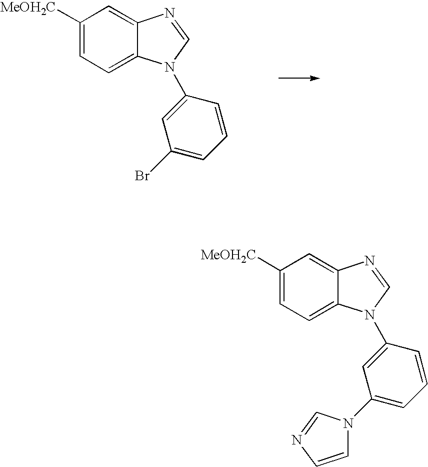
1-(3-(1-Imidazolyl)phenyl)-5-(methoxymethyl)benzimidazole. A mixture of 1-(3-bromophenyl)-5-(methoxymethyl)benzimidazole (1 g, 3.2 mmol), imidazole (0.32 g; 5.5 mmol), potassium carbonate (0.44 g, 3.16 mmol) and a catalytic amount of copper in N-methyl-2-pyrrolidinone (5 ml) was heated to 140° C. overnight. The cooled reaction mixture was poured into water and stirred. The precipitate was filtered off and extracted with a hot mixture of ethanol and dichloromethane. The extract was evaporated to dryness and the residue (1 g) was recrystallised from ethanol (20 ml) to leave the pure product (0.6 g; 62.5%). Mp. 178.3-179.6° C.
1-(3-(1-Imidazolyl)phenyl)-5-(ethoxymethyl)benzimidazole is prepared analogously from 1-(3-bromophenyl)-5-(ethoxymethyl)benzimidazole.
1-(3-(1-Imidazolyl)phenyl)-5-(isopropoxymethyl)benzimidazole is prepared analogously from 1-(3-bromophenyl)-5-(isopropoxymethyl)benzimidazole.
1-(3-(1-Imidazolyl)phenyl)-5-(propoxymethyl)benzimidazole is prepared analogously from 1-(3-bromophenyl)-5-(propoxymethyl)benzimidazole.
1-(3-(1-Imidazolyl)phenyl)-5-(cyclopropylmethoxymethyl)benzimidazole is prepared analogously from 1-(3-bromophenyl)-5-(cyclopropylmethoxymethyl)-benzimidazole.
Example 2 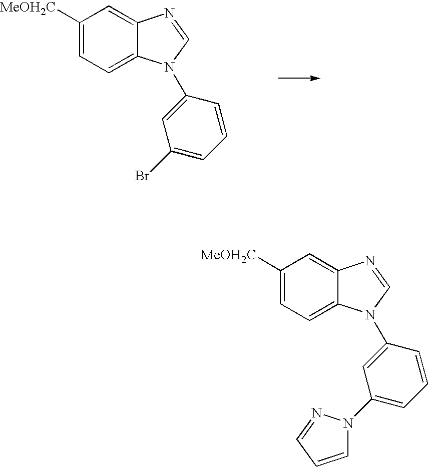
1-(3-(1-Pyrazolyl)phenyl)-5-(methoxymethyl)benzimidazole was prepared analogously from pyrazol and 1-(3-bromophenyl)-5-(methoxymethyl)benzimidazole. Yield 10%. Mp. 126-128° C.
1-(3-(1-Pyrazolyl)phenyl)-5-(hydroxymethyl)benzimidazole is prepared analogously from 1-(3-bromophenyl)-5-(hydroxymethyl)benzimidazole.
1-(3-(1-Pyrazolyl)phenyl)-5-(ethoxymethyl)benzimidazole is prepared analogously from 1-(3-bromophenyl)-5-(ethoxymethyl)benzimidazole.
1-(3-(1-Pyrazolyl)phenyl)-5-(isopropoxymethyl)benzimidazole is prepared analogously from 1-(3-bromophenyl)-5-(isopropoxymethyl)benzimidazole.
1-(3-(1-Pyrazolyl)phenyl)-5-(propoxymethyl)benzimidazole is prepared analogously from 1-(3-bromophenyl)-5-(propoxymethyl)benzimidazole.
1-(3-(1-Pyrazolyl)phenyl)-5-(cyclopropylmethoxymethyl)benzimidazole is prepared analogously from 1-(3-bromophenyl)-5-(cyclopropylmethoxymethyl)benzimidazole.
Example 3 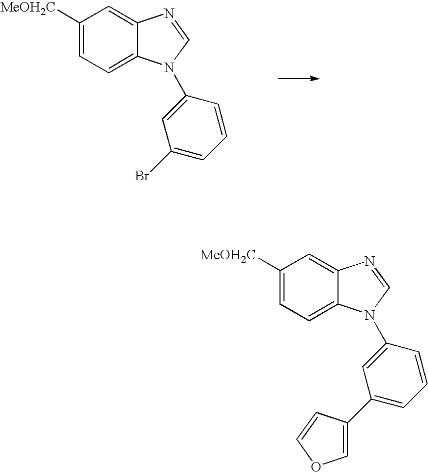
1-(3-(3-Furyl)phenyl)-5-(methoxymethyl)benzimidazole. A mixture of 1-(3-bromophenyl)-5-(methoxymethyl)benzimidazole (0.50 g; 1.58 mmol), 3-furyl boronic acid (0.17 g; 1.58 mmol), sodium bicarbonate (0.66 g; 7.86 mmol) and a catalytic amount of tetrakis(triphenylphosphine)palladium in a mixture of dimethoxyethane (5 ml) and water (2.5 ml) was heated to 80° C. in a nitrogen atmosphere overnight. The cooled mixture was poured into ice-water a extracted with ethyl acetate. The organic extract was dried over magnesium sulfate and concentrated under reduced pressure. The concentrate was purified by column chromatography on silica gel using a mixture of ethyl acetate and petroleum ether (9:1, v/v) as the eluent.
Yield: 0.22 g (46%). Mp 92-94° C.
1-(3-(3-Furyl)phenyl)-5-(hydroxymethyl)benzimidazole is prepared analogously from 1-(3-bromophenyl)-5-(hydroxymethyl)benzimidazole.
1-(3-(3-Furyl)phenyl)-5-(ethoxymethyl)benzimidazole is prepared analogously from 1-(3-bromophenyl)-5-(ethoxymethyl)benzimidazole.
1-(3-(3-Furyl)phenyl)-5-(isopropoxymethyl)benzimidazole is prepared analogously from 1-(3-bromophenyl)-5-(isopropoxymethyl)benzimidazole.
1-(3-(3-Furyl)phenyl)-5-(propoxymethyl)benzimidazole is prepared analogously from 1-(3-bromophenyl)-5-(propoxymethyl)benzimidazole.
1-(3-(3-Furyl)phenyl)-5-(cyclopropylmethoxymethyl)benzimidazole is prepared analogously from 1-(3-bromophenyl)-5-(cyclopropylmethoxymethyl)benzimidazole. 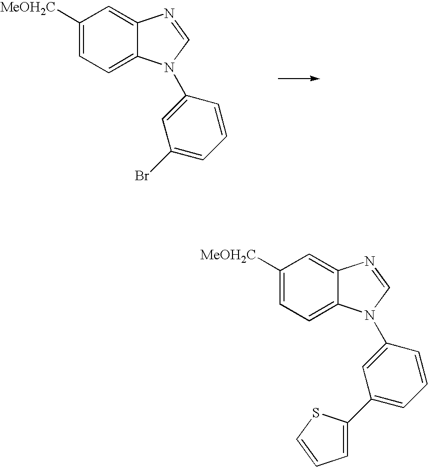
1-(3-(2-Thienyl)phenyl)-5-(methoxymethyl)benzimidazole. A mixture of 1-(3-bromophenyl)-5-(methoxymethyl)benzimidazole (1.0 g; 3.16 mmol), tributyltin (1.77 g; 4.75 mmol) and dichloro bis(triphenylphosphine)palladium (30 mg) in anhydrous dimethyl formamide (10 ml) was heated to 80° C. overnight. The cooled mixture was poured into water (50 ml) and extracted with ethyl acetate. The organic extract was dried over magnesium sulfate and concentrated under reduced pressure. The concentrate was eluted through silica gel with ethyl acetate. The pure fractions were collected and evaporated under reduced pressure. The product precipitated from the residue upon trituation with a mixture of petroleum ether and diethyl ether (1:1, v/v).
Yield 0.34 g (34%). Mp 69-71° C.
1-(3-(2-Thienyl)phenyl)-5-(hydroxymethyl)benzimidazole is prepared analogously from 1-(3-bromophenyl)-5-(hydroxymethyl)benzimidazole.
1-(3-(2-Thienyl)phenyl)-5-(ethoxymethyl)benzimidazole is prepared analogously from 1-(3-bromophenyl)-5-(ethoxymethyl)benzimidazole.
1-(3-(2-Thienyl)phenyl)-5-(isopropoxymethyl)benzimidazole is prepared analogously from 1-(3-bromophenyl)-5-(isopropoxymethyl)benzimidazole.
1-(3-(2-Thienyl)phenyl)-5-(propoxymethyl)benzimidazole is prepared analogously from 1-(3-bromophenyl)-5-(propoxymethyl)benzimidazole.
1-(3-(2-Thienyl)phenyl)-5-(cyclopropylmethoxymethyl)benzimidazole is prepared analogously from 1-(3-bromophenyl)-5-(cyclopropylmethoxymethyl)benzimidazole. 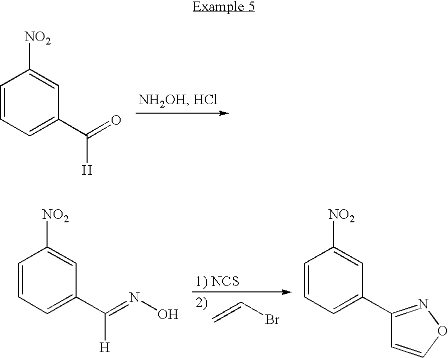
3-Nitrobenzaldehyde oxime. To a suspension of 3-nitrobenzaldehyde (50.5 g, 0.33 mol) in abs. ethanol (500 ml) was added hydroxylamine, hydrochloride (34.8 g, 0.50 mol) and triethylamine (46.5 ml, 0.33 mol) and the resultant mixture was stirred at reflux over night. The solvent was distilled off under reduced pressure, and water was added to the residue. The resultant solution was rendered alkaline by addition of saturated, aqueous sodium carbonate and the precipitate was filtered off, washed with water and air-dried to leave the product (51.7 g).
3-(3-Nitrophenyl)isoxazole. To a solution of the above product (10.0 g, 60.2 mmol) in anhydrous dimethyl formamide (500 ml) was added N-chlorosuccinimide (9.6 g, 72.3 mmol) and the resultant mixture was stirred at 60° C. for 3 hours. The mixture was cooled in an ice-bath and vinyl bromide (40 ml, 0.57 mol) was added dropwise. The resultant solution was maintained at 0° C. while a solution of triethyl amine (42 ml, 0.3 mol) in anhydrous dimethyl formamide was added cautiously over 2 hours. After the addition the mixture was stirred at ambient temperature over night. The solvent was distilled off under reduced pressure and the residue was partitioned between water and ethyl acetate. The layers were separated and the organic layer was washed with brine, dried over sodium sulphate and evaporated to dryness. The residue was purified by column chromatography on silica gel using a mixture of ethyl acetate and petroleum ether (1:9, v/v) as the eluent to leave the product (7.1 g). 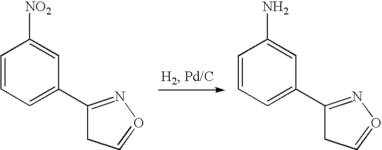
3-(3-Aminophenyl)isoxazole. A suspension of the above product (7.1 g, 37.4 mmol) in abs. ethanol (100 ml) was hydrogenated at ambient pressure, using Pd (5% on activated carbon) as the catalyst, until the hydrogen uptake had ceased. The resultant mixture was filtered through celite and the filtrate was evaporated to leave the desired product as a yellow oil (5.75 g). 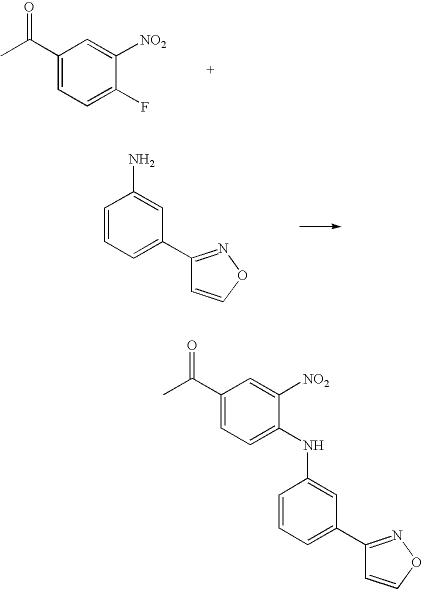
4-Acetyl-2-nitro-N-(3-(3-isoxazolyl)phenyl)aniline. A mixture of 4-fluoro-3-nitroacetophenone (3.0 g, 18.7 mmol) and 3-(3-aminophenyl)isoxazole in anhydrous N-methyl 2-pyrrolidinone (30 ml) was stirred at 40° C. over night and then poured into ice-water. The oily precipitate was isolated and recrystallised from ethanol (100 ml) to yield the desired product (3.5 g). 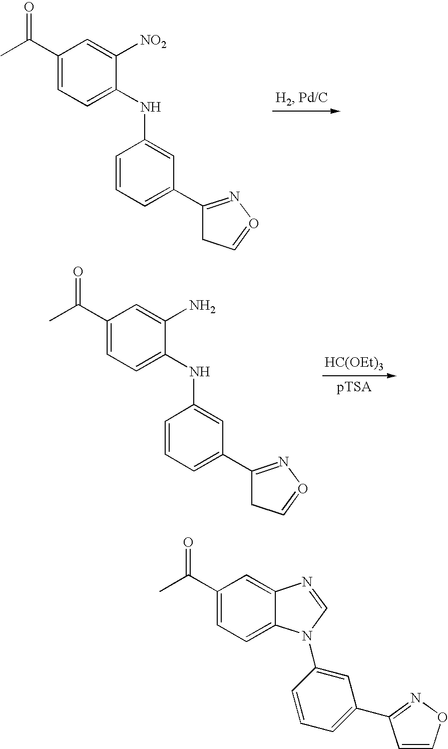
4-Acetyl-2-amino-N-(3-(3-isoxazolyl)phenyl)aniline. To a solution of the above product (23.8 g), in abs. ethanol (200 ml) was added Pd (2.4 g, 5% on activated carbon) and the resultant mixture was hydrogenated at ambient pressure until the hydrogen uptake had ceased. The mixture was filtered through celite and the product was isolated from the filtrate by evaporation under reduced pressure (16.0 g).
5-Acetyl-1-(3-(3-isoxazolyl)phenyl)benzimidazole. To a solution of the above product (16.0 g, 51.5 mmol) in tetrahydrofurane (150 ml) was added triethyl orthoformate (11.8 ml, 70.9 mmol) and p-toluenesulphonic acid (catalytic amounts) and the resultant mixture was stirred at reflux for 30 min. After cooling, the mixture was concentrated under reduced pressure and the product precipitated from the residue upon addition of dichloromethane. Filtration and air-drying afforded the desired product (12.0 g). 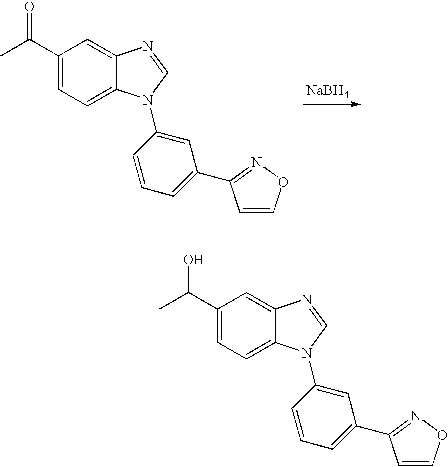
5-(1-Hydroxyethyl)-1-(3-(3-isoxazolyl)phenyl)benzimidazole. A suspension of the above product (12.0 g, 39.6 mmol) in a mixture of dimethylformamide (270 ml) and methanol (30 ml) was stirred at 60° C. in a nitrogen atmosphere. Sodium borohydride (1.5 g, 39.6 mmol) was added in portions and stirring was continued at 60° C. for 1 hour, whereafter the resultant solution was left with stirring at ambient temperature over night. The resultant mixture was diluted with four volumes of water and extracted with ethyl acetate. The organic extract was concentrated under reduced pressure. The concentrate was diluted with diethyl ether, washed with aqueous calcium chloride (3M), dried over magnesium sulphate and evaporated to dryness to leave the desired product (11.5 g). Mp 147-151° C. 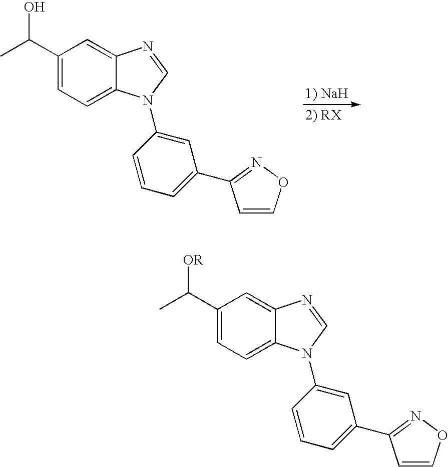
5-(1-(Allyloxy)ethyl)-1-(3-(3-isoxazolyl)phenyl)benzimidazole. To a cooled (0° C.) suspension of the above product (0.6 g, 1.97 mmol) in a mixture of dimethoxyethane (10 ml) and dimethyl formamide (5 ml) was added sodium hydride (0.16 g 60% dispersion in mineral oil, 3.93 mmol). The resultant mixture was stirred at 0° C. until the evolution of hydrogen had ceased. Allylbromide (0.26 ml, 2.95 mmol) was added and the reaction mixture was stirred at ambient conditions over night, whereafter it was partitioned between water and ethyl acetate. The organic extract was washed with aqueous calcium chloride (3M), dried over sodium sulphate and concentrated under reduced pressure. The concentrate was eluted through silica gel with a mixture of ethyl acetate and petroleum ether (1:1, v/v) to afford the pure product (0.25 g). Mp. 86-93° C.
5-(1-(Propargyloxy)ethyl)-1-(3-(3-isoxazolyl)phenyl)benzimidazole was prepared analogously using propargylbromide. Mp 70-80° C.
5-(1-Methoxyethyl)-1-(3-(3-isoxazolyl)phenyl)benzimidazole was prepared analogously using iodomethane. The product was isolated as the hydrochloride m/z 320.1 (M+1).
5-(1-Ethoxyethyl)-1-(3-(3-isoxazolyl)phenyl)benzimidazole is prepared analogously using iodoethane.
5-(1-Propoxyethyl)-1-(3-(3-isoxazolyl)phenyl)benzimidazole is prepared analogously using 1-bromopropane.
5-(1-Isopropoxyethyl)-1-(3-(3-isoxazolyl)phenyl)benzimidazole is prepared analogously using 2-bromopropane.
5-(1-(Cyclopropylmethoxy)ethyl)-1-(3-(3-isoxazolyl)phenyl)benzimidazole is prepared analogously using bromomethylcyclopropane. 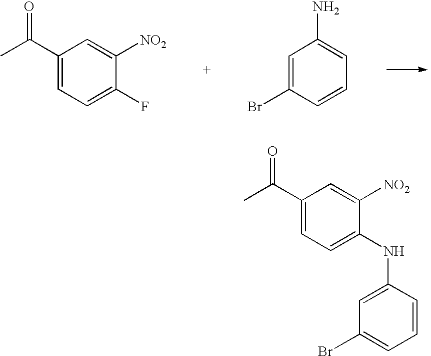
4-Acetyl-N-(3-bromophenyl)-2-nitroaniline. A mixture of 4-fluoro-3-nitroacetophenone (10.0 g, 54.6 mmol) and 3-bromoaniline (14.9 ml, 0.14 mol) was stirred at 60° C. for two days. The resultant reaction mixture was partitioned between diluted hydrochloric acid and dichloromethane. The organic layer was washed with water, dried over magnesium sulphate and evaporated under reduced pressure. The residue was recrystallised from ethanol to afford the desired product (12.0 g). 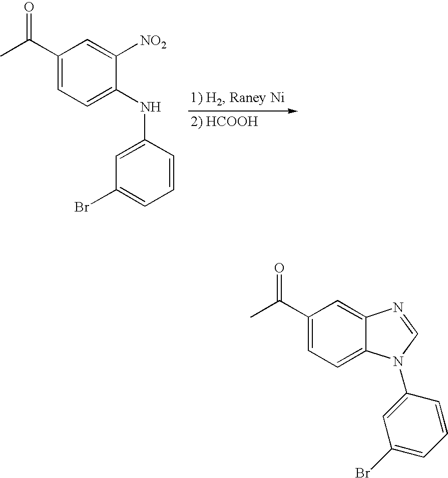
4-Acetyl-2-amino-N-(3-bromophenyl)aniline. To a suspension of the above product (10.0 g, 30.0 mmol) in a mixture of abs. ethanol (100 ml) and dichloromethane (25 ml) was added Raney nickel (0.5 g) and the resultant mixture was hydrogenated until the hydrogen uptake had ceased. To the resultant suspension was added dichloromethane to afford a solution. This solution was filtered through celite, and the filtrate was evaporated under reduced pressure to afford the desired product (8.05 g).
5-Acetyl-1-(3-bromophenyl)benzimidazole. The above product (8.05 g, 26.4 mmol) was stirred in formic acid (50 ml) at 80° C. for 1.5 hours. After cooling, the mixture was poured into water (200 ml) and the precipitated product was filtered off, washed with water and air-dried to leave 8.08 g. 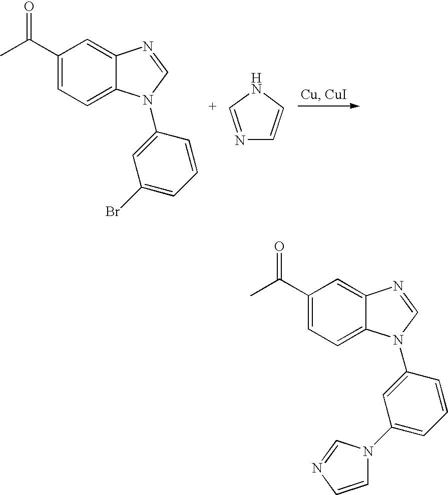
5-Acetyl-1-(3-(1-imidazolyl)phenyl)benzimidazole. A mixture of the above product (6.5 g, 20.6 mmol), imidazole (4.21 g, 61.9 mmol), potassium carbonate (2.85 g, 20.6 mmol) and catalytic amounts of copper and copper(1)iodide in N-methyl-2-pyrrolidinone (10 ml) was stirred under nitrogen at 180° C. for 2 hours and then left to cool at ambient conditions over night. The resultant reaction cake was extracted with a mixture of dichloromethane and methanol. The extract was concentrated under reduced pressure and the concentrate was triturated in ethyl acetate to afford the desired product (6.01 g). 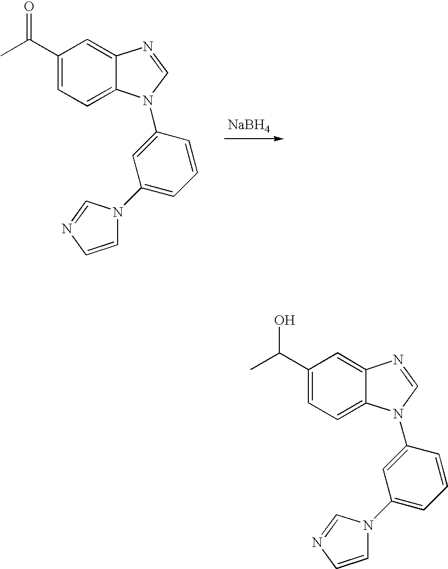
5-(1-Hydroxyethyl)-1-(3-(1-imidazolyl)phenyl)benzimidazole was prepared form the above product analogously to the procedure described in Example 5. Mp 146-148° C.
5-(1-Methoxyethyl)-1-(3-(1-imidazolyl)phenyl)benzimidazole is prepared by methylation of the above product analogously to the procedure described in Example 5.
5-(1-Ethoxyethyl)-1-(3-(1-imidazolyl)phenyl)benzimidazole is prepared analogously, by ethylation with iodoethane.
5-(1-Propoxyethyl)-1-(3-(1-imidazolyl)phenyl)benzimidazole is prepared analogously, by alkylation with 1-bromopropane.
5-(1-Isopropoxyethyl)-1-(3-(1-imidazolyl)phenyl)benzimidazole is prepared analogously, by alkylation with 2-bromopropane.
5-(1-(Cyclopropylmethoxy)ethyl)-1-(3-(1-imidazolyl)phenyl)benzimidazole is prepared analogously, by alkylation with bromomethylcyclopropane. 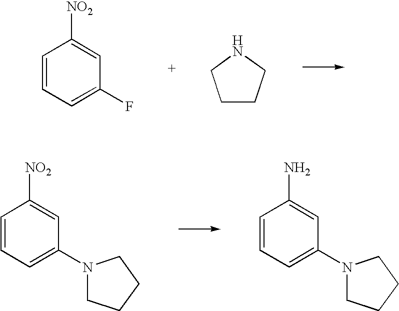
1-(3-Nitrophenyl)pyrrolidine. A mixture of 3-fluoronitrobenzene (21.2 ml, 0.20 mol) and pyrrolidine (33.4 ml, 0.40 mol) was stirred at reflux over night. The cooled mixture was poured into water and the precipitate was filtered off, washed with water and air-dried to afford to desired product, quantitatively.
3-(1-Pyrrolidinyl)aniline. To a suspension of the above product (38.0 g, 0.20 mol) in a mixture of methanol (100 ml) and dichloromethane (50 ml) was added Pd (3.0 g, 5% on activated carbon) and the resultant mixture was hydrogenated until the hydrogen uptake had ceased. Filtration through celite and removal of solvent from the filtrate left the desired product, quantitatively. 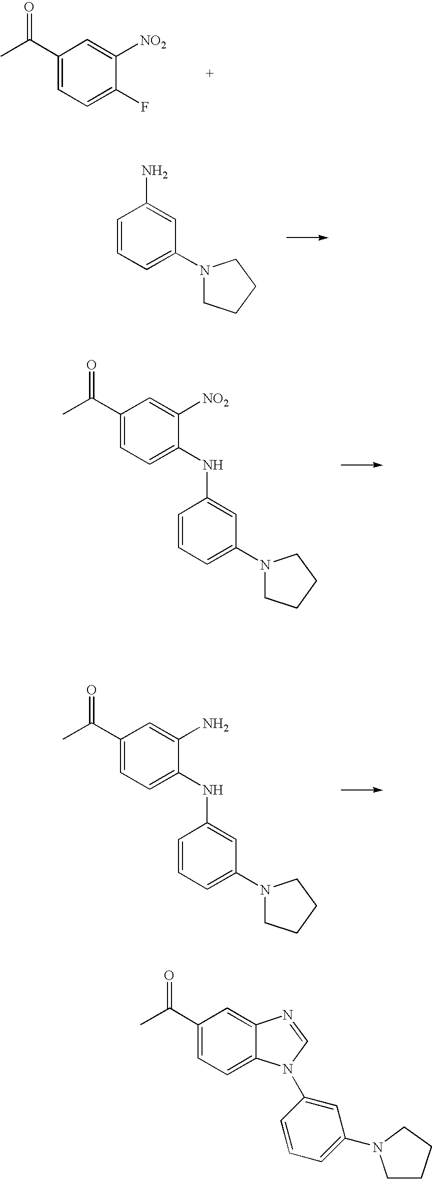
5-Acetyl-1-(3-(1-pyrrolidinyl)phenyl)benzimidazole was prepared from the above product and 4-fluoro-3-nitroacetophenone by a reaction sequence analogous to that described in Example 5.
5-(1-Hydroxyethyl)-1-(3-(1-pyrrolidinyl)phenyl)benzimidazole is prepared by reduction of the above product with sodium borohydride as described in Example 5.
5-(1-Methoxyethyl)-1-(3-(1-pyrrolidinyl)phenyl)benzimidazole is prepared by alkylation of the above product with iodomethane as described in Example 5.
5-(1-Ethoxyethyl)-1-(3-(1-pyrrolidinyl)phenyl)benzimidazole is prepared analogously, by ethylation with iodoethane.
5-(1-Propoxyethyl)-1-(3-(3-(1-pyrrolidinyl)phenyl)benzimidazole is prepared analogously, by alkylation with 1-bromopropane.
5-(1-Isopropoxyethyl)-1-(3-(1-pyrrolidinyl)phenyl)benzimidazole is prepared analogously, by alkylation with 2-bromopropane to the procedure.
5-(1-Cyclopropylmethoxy)ethyl)-1-(3-(1-pyrrolidinyl)phenyl)benzimidazole is prepared analogously, by alkylation with bromomethylcyclopropane. 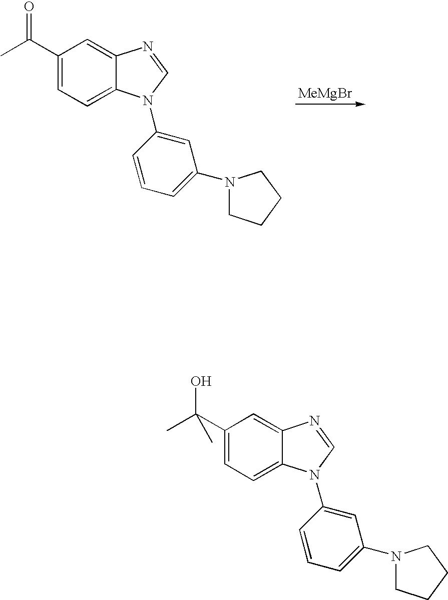
5-(2-Hydroxy-2-propyl)-1-(3-(1-pyrrolidinyl)phenyl)benzimidazole. To a stirred suspension of 5-acetyl-1-(3-(1-pyrrolidinyl)phenyl)benzimidazole (5.2 g, 16.9 mmol) in anhydrous tetrahydrofurane (25 ml) was added methyl magnesium bromide (17 ml, 3M in diethyl ether) dropwise over 20 min. in a nitrogen atmosphere. After the addition, the resultant mixture was stirred for additionally 30 min, whereafter saturated, aqueous ammonium chloride (15 ml) was added. The volatile solvent was removed by evaporation. Dichloromethane was added and the layers were separated. Three successive extractions were performed and the combined organic extracts were washed with water, dried over magnesium sulphate and concentrated under reduced pressure. The concentrate was purified by column chromatography on silica gel using ethyl acetate as the eluent to afford the desired product (3.3 g). Mp 167-171° C.
5-(2-Methoxy-2-propyl)-1-(3-(1-pyrrolidinyl)phenyl)benzimidazole. This was prepared from the above product by alkylation with iodomethane in analogy with Example 5. m/z 336.2 (M+1).
5-(2-Ethoxy-2-propyl)-1-(3-(1-pyrrolidinyl)phenyl)benzimidazole is prepared analogously, by ethylation with iodoethane.
5-(2-Propoxy-2-propyl)-1-(3-(1-pyrrolidinyl)phenyl)benzimidazole is prepared analogously, by alkylation with 1-bromopropane.
5-(2-Isopropoxy-2-propyl)-1-(3-(1-pyrrolidinyl)phenyl)benzimidazole is prepared analogously, by alkylation with 2-bromopropane.
5-(2-(Cyclopropylmethoxy)-2-propyl)-1-(3-(1-pyrrolidinyl)phenyl)benzimidazole is prepared analogously, by alkylation with bromomethylcyclopropane. 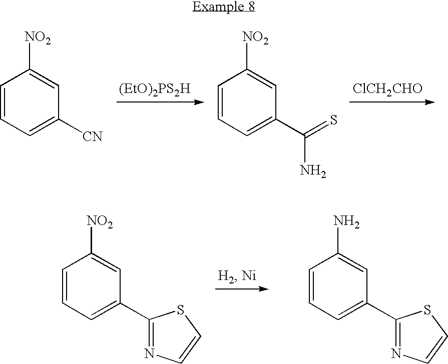
3-Nitro-thio-benzamide. To a stirred solution of 3-nitrobenzonitril (14.5 g, 98 mmol) and diethyl dithiophosphate (16.5 ml, 98 mmol) in ethyl acetate (200 ml) was led gaseous hydrogen chloride. When the evolution of heat had ceased the gas inlet was disconnected and the resultant mixture was left with stirring at ambient temperature over night. Saturated, aqueous sodium carbonate (400 ml) was added, and the layers were separated. The organic layer was dried over magnesium sulphate and concentrated under reduced pressure. The desired product precipitated from the concentrate upon trituration with petroleum ether to afford 17.74 g.
2-(3-Nitrophenyl)thiazole. To a suspension of the above product (17.74 g, 97 mmol) in glacial acetic acid (150 ml) was added chloroacetaldehyde (12.7 ml, 100 mmol) and the resultant mixture was stirred at 125° C. for 2 hours. The cooled mixture was poured into ice-water and rendered alkaline by addition of aqueous sodium hydroxide (12M). Ethyl acetate was added, and the resultant emulsion was filtered through celite prior to separation of the layers. The aqueous layer was extracted 3 times with ethyl acetate, and the combined organic layers were dried over magnesium sulphate and concentrated under reduced pressure. The concentrate was purified by column chromatography on silica gel eluting with a mixture of ethyl acetate and ligroin (1:1 v/v) to afford the desired product (6.6 g).
3-(2-Thiazolyl)aniline. To a suspension of the above product (6.1 g, 29.6 mmol) in ethanol (170 ml) was added Raney nickel (0.5 g) and the resultant mixture was hydrogenated at ambient pressure until the hydrogen uptake had ceased. Filtration through celite and evaporation of the solvent from the filtrate left the desired product, quantitatively. 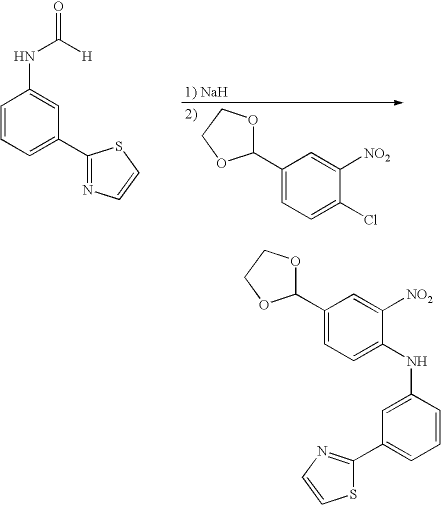
N-(3-(2-Thiazolyl)phenyl)formamide. The above product (2.7 g) was treated with formic acid (10 ml) at reflux for 30 min. Excess of formic acid was removed by evaporation under reduced pressure and the residue was partioned between saturated, aqueous sodium carbonate and ethyl acetate. The organic phase was dried over sodium sulphate and evaporated to dryness to leave the desired product (3.1 g).
4-(2-Dioxolanyl)-2-nitro-N-(3-(2-thiazolyl)phenyl)aniline. To a solution of the above product (3.1 g, 15.2 mmol) in anhydrous dimethyl formamide (30 ml) was added sodium hydride (0.73 g 60% dispersion in mineral oil, 18.2 mmol). When the evolution of hydrogen had ceased, 2-(4-chloro-3-nitrophenyl)dioxolane (3.5 g, 15.2 mmol) was added and the resultant mixture was stirred at 120° C. over night. The cooled mixture was poured into ice-water (300 g) and extracted with ethyl acetate. The organic extract was washed with aqueous calcium chloride (3M), dried over magnesium sulphate and concentrated under reduced pressure. The concentrate was triturated in diethyl ether to leave the desired product as a reddish solid (3.6 g).
4-(2-Dioxolanyl)-2-nitro-N-(3-(1-imidazolyl)phenyl)aniline was prepared analogously from 2-(4-chloro-3-nitrophenyl)dioxolane and N-(3-(1-imidazolyl)phenyl)-formamide.
4-(2-Dioxolanyl)-2-nitro-N-(3-(3-isoxazolyl)phenyl)aniline is prepared analogously from 2-(4-chloro-3-nitrophenyl)dioxolane and N-(3-(3-isoxazolyl)phenyl)formamide.
4-(2-Dioxolanyl)-2-nitro-N-(3-(1-pyrrolyl)phenyl)aniline is prepared analogously from 2-(4-chloro-3-nitrophenyl)dioxolane and N-(3-(1-pyrrololyl)phenyl)formamide. 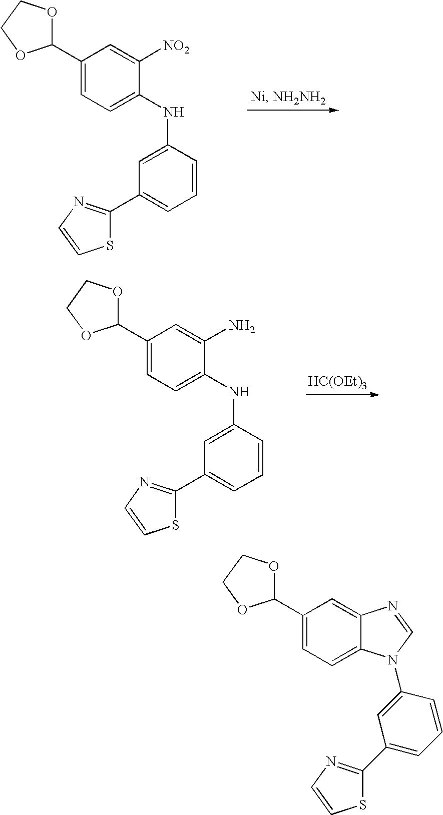
2-Amino-4-(2-dioxolanyl)-N-(3-(2-thiazolyl)phenyl)aniline. To a solution of 4-(2-dioxolanyl)-2-nitro-N-(3-(2-thiazolyl)phenyl)aniline (3.6 g, 9.7 mmol) in a mixture of tetrahydrofurane (100 ml) and abs. ethanol (50 ml) was added hydrazine, hydrate (1.25 ml, 38.9 mmol) and a catalytic amount of Raney nickel. The resultant mixture was stirred under nitrogen at room temperature for 1 hour and then filtered through celite. The filtrate was evaporated to dryness to afford the desired product, quantitatively, as a brown oil.
2-Amino-4-(2-dioxolanyl)-N-(3-(1-imidazolyl)phenyl)aniline was prepared analogously from 4-(2-dioxolanyl)-2-nitro-N-(3-(1-imidazolyl)phenyl)aniline.
2-Amino-4-(2-dioxolanyl)-N-(3-(3-isoxazolyl)phenyl)aniline is prepared analogously from 4-(2-dioxolanyl)-2-nitro-N-(3-(3-isoxazolyl)phenyl)aniline.
2-Amino-4-(2-dioxolanyl)-N-(3-(1-pyrrolyl)phenyl)aniline is prepared analogously from 4-(2-dioxolanyl)-2-nitro-N-(3-(1-pyrrolyl)phenyl)aniline.
5-(2-Dioxolanyl)-1-(3-(2-thiazolyl)phenyl)benzimidazole was prepared from 2-amino-4-(2-dioxolanyl)-N-(3-(2-thiazolyl)phenyl)aniline (3.3 g, 9.7 mmol) and triethyl orthoformate (3.2ml, 19.4 mmol) using p-toluenesulphonic acid as the catalyst as described in Example 5 to afford 2.5 g.
5-(2-Dioxolanyl)-1-(3-(1-imidazolyl)phenyl)benzimidazole was prepared analogously from 2-amino-4-(2-dioxolanyl)-N-(3-(1-imidazolyl)phenyl)aniline.
5- (2-Dioxolanyl)-1-(3-(3-isoxazolyl)phenyl)benzimidazole is prepared analogously from 2-amino-4-(2-dioxolanyl)-N-(3-(3-isoxazolyl)phenyl)aniline.
5- (2-Dioxolanyl)-1-(3-(1-pyrrolyl)phenyl)benzimidazole is prepared analogously from 2-amino-4-(2-dioxolanyl)-N-(3-(1-pyrrolyl)phenyl)aniline. 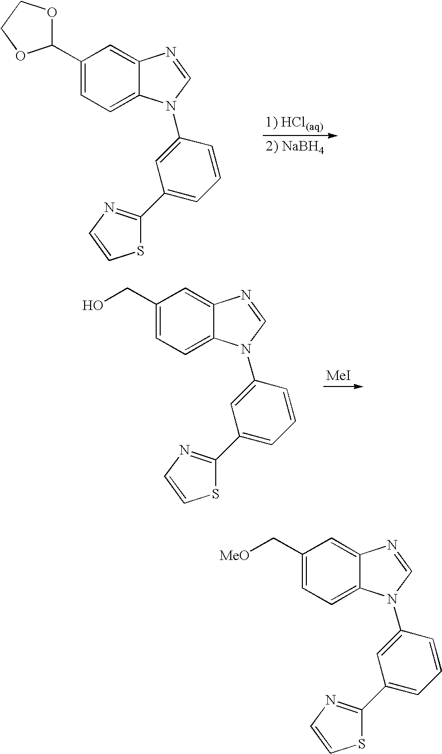
5-Formyl-1-(3-(2-thiazolyl)phenyl)benzimidazole. A solution of 5-(2-dioxolanyl)-1-(3-(2-thiazolyl)phenyl)benzimidazole (2.5 g, 7.14 mmol) in hydrochloric acid (30 ml, 1M) was stirred at ambient conditions for 15 min. The resultant solution was cooled in ice and rendered alkaline by addition of saturated, aqueous sodium carbonate. The precipitate was filtered off, washed with water and air-dried to afford the desired product as yellow crystals (2.1 g).
5-Formyl-1-(3-(1-imidazolyl)phenyl)benzimidazole was prepared analogously from 5-(2-dioxolanyl)-1-(3-(1-imidazolyl)phenyl)benzimidazole.
5-Formyl-1-(3-(3-isoxazolyl)phenyl)benzimidazole is prepared analogously from 5-(2-dioxolanyl)-1-(3-(3-isoxazolyl)phenyl)benzimidazole
5-Formyl-1-(3-(1-pyrrolyl)phenyl)benzimidazole is prepared analogously from 5-(2-dioxolanyl)-1-(3-(1-pyrrolyl)phenyl)benzimidazole
5-(Hydroxymethyl)-1-(3-(2-thiazolyl)phenyl)benzimidazole. 5-Formyl-1-(3-(2-thiazolyl)phenyl)benzimidazole (1.0 g, 3.27 mmol) was treated with sodium borohydride (0.12 g, 3.27 mmol) as described in Example 5 (1.0 g) Mp 115-118° C.
5-(Hydroxymethyl)-1-(3-(1-imidazolyl)phenyl)benzimidazole was prepared analogously from 5-formyl-1-(3-(1-imidazolyl)phenyl)benzimidazole Mp 273-274° C.
5-(Hydroxymethyl)-1-(3-(3-isoxazolyl)phenyl)benzimidazole is prepared analogously from 5-formyl-1-(3-(3-isoxazolyl)phenyl)benzimidazole
5-(Hydroxymethyl)-1-(3-(1-pyrrolyl)phenyl)benzimidazole is prepared analogously from 5-formyl-1-(3-(1-pyrrolyl)phenyl)benzimidazole
5-(Methoxymethyl)-1-(3-(2-thiazolyl)phenyl)benzimidazole. 5-Hydroxymethyl-1-(3-(2-thiazolyl)phenyl)benzimidazole (1.0 g, 3.26 mmol) was methylated with iodomethane as described in Example 5 (0.54 g). The product was isolated as the hydrochloride. Mp 175-178° C.
5-(Methoxymethyl)-1-(3-(3-isoxazolyl)phenyl)benzimidazole is prepared analogously from 5-hydroxymethyl-1-(3-(3-isoxazolyl)phenyl)benzimidazole.
5-(Ethoxymethyl)-1-(3-(3-isoxazolyl)phenyl)benzimidazole is prepared analogously by ethylation with iodoethane.
5-(Isopropoxymethyl)-1-(3-(3-isoxazolyl)phenyl)benzimidazole is prepared analogously by alkylation with 2-bromopropane.
5-(Propoxymethyl)-1-(3-(3-isoxazolyl)phenyl)benzimidazole is prepared analogously by alkylation with 1-bromopropane.
5-(Cyclopropylmethoxymethyl)-1-(3-(3-isoxazolyl)phenyl)benzimidazole is prepared analogously by alkylation with bromomethylcyclopropane.
5-(Methoxymethyl)-1-(3-(1-pyrrolyl)phenyl)benzimidazole is prepared analogously from 5-hydroxymethyl-1-(3-(1-pyrrolyl)phenyl)benzimidazole.
5-(Ethoxymethyl)-1-(3-(1-pyrrolyl)phenyl)benzimidazole is prepared analogously by ethylation with iodoethane.
5-(Isopropoxymethyl)-1-(3-(1-pyrrolyl)phenyl)benzimidazole is prepared analogously by alkylation with 2-bromopropane.
5-(Propoxymethyl)-1-(3-(1-pyrrolyl)phenyl)benzimidazole is prepared analogously by alkylation with 1-bromopropane.
5-(Cyclopropylmethoxymethyl)-1-(3-(1-pyrrolyl)phenyl)benzimidazole is prepared analogously by alkylation with bromomethylcyclopropane.
5-(Ethoxymethyl)-1-(3-(2-thiazolyl)phenyl)benzimidazole is prepared analogously by ethylation with iodoethane.
5-(Isopropoxymethyl)-1-(3-(2-thiazolyl)phenyl)benzimidazole is prepared analogously by alkylation with 2-bromopropane.
5-(Propoxymethyl)-1-(3-(2-thiazolyl)phenyl)benzimidazole is prepared analogously by alkylation with 1-bromopropane.
5-(Cyclopropylmethoxymethyl)-1-(3-(2-thiazolyl)phenyl)benzimidazole is prepared analogously by alkylation with bromomethylcyclopropane.

### TEST METHODS

Test Method 1

In Vitro Inhibition of ³H-flunitrazepam (³H-FNM) Binding

The GABA recognition site and the benzodiazepine modulatory unit can selectively be labelled with ³H-flunitrazepam.

Tissue Preparation

Preparations are performed at 0-4° C. unless otherwise indicated. Cerebral cortex from male Wistar rats (150-200 g) is homogenised for 5-10 sec in 20 ml Tris-HCl (30 mM, pH 7.4) using an Ultra-Turrax homogeniser. The suspension is centrifuged at 27,000×g for 15 min and the pellet is washed three times with buffer (centrifuged at 27,000×g for 10 min). The washed pellet is homogenized in 20 ml of buffer and incubated on a water bath (37° C.) for 30 min to remove endogenous GABA and then centrifuged for 10 min at 27,000×g. The pellet is then homogenized in buffer and centrifuged for 10 min at 27,000×g. The final pellet is resuspended in 30 ml buffer and the preparation is frozen and stored at −20° C.

Assay

The membrane preparation is thawed and centrifuged at 2° C. for 10 min at 27,000×g. The pellet is washed twice with 20 ml 50 mM Tris-citrate, pH 7.1 using an Ultra-Turrax homogeniser and centrifuged for 10 min at 27,000×g. The final pellet is resuspended in 50 mM Tris-citrate, pH 7.1 (500 ml buffer per g of original tissue), and then used for binding assays. Aliquots of 0.5 ml tissue are added to 25 μl of test solution and 25 μl of ³H-FNM (1 nM, final concentration), mixed and incubated for 40 min at 2° C. Non-specific binding is determined using Clonazepam (1 μM, final concentration). After incubation the samples are added 5 ml of ice-cold buffer and poured directly onto Whatman GF/C glass fibre filters under suction and immediately washed with 5 ml ice-cold buffer. The amount of radioactivity on the filters is determined by conventional liquid scintillation counting. Specific binding is total binding minus non-specific binding.

Results

25-75% inhibition of specific binding must be obtained, before calculation of an IC₅₀.

The test value will be given as IC₅₀ (the concentration (μM) of the test substance which inhibits the specific binding of ³H-FNM by 50%).

$${IC}_{50} = ( applied test substance concentration , μM ) \times \frac{1}{( \frac{{C}_{o}}{{C}_{x}} - 1 )}$$

where

Cₒ is specific binding in control assays, and

Cₓ is the specific binding in the test assay.

(The calculations assume normal mass-action kinetics).

1. A compound of general formula (I): 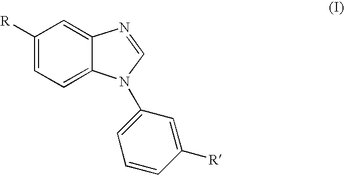
or an N-oxide thereof, or any of its isomers or any mixture of its isomers,
or a pharmaceutically acceptable salt thereof,
wherein
R represents hydroxyalkyl, alkoxyalkyl, cycloalkoxyalkyl, cycloalkylalkoxyalkyl, alkenyloxyalkyl, or alkynyloxyalkyl; and
R′ represents a 5-membered heterocyclic ring;
which heterocyclic ring may optionally be substituted with one or more substituents independently selected from the group consisting of halo, hydroxy, amino, alkylamino, aminoalkyl, cyano, nitro, trifluoromethyl, trifluoromethoxy, alkoxy, cycloalkoxy, alkyl, cycloalkyl, cycloalkylalkyl, alkenyl, and alkynyl.

2. The compound of claim 1, wherein R represents hydroxymethyl, alkoxymethyl, or cycloalkylalkoxymethyl.

3. The compound of claim 1, wherein R represents 1-hydroxyethyl, 1-alkoxyethyl, 1-(cycloalkylalkoxy)ethyl, 1-(alkenyloxy)ethyl, or 1-(alkynyloxy)ethyl.

4. The compound of claim 1, wherein R represents 2-hydroxy-2-propyl, 2-alkoxy-2-propyl, or 2-(cycloalkylalkoxy)-2-propyl.

5. The compound of claim 1, wherein R′ represents imidazolyl, pyrazolyl, thienyl, furyl, isoxazolyl, thiazolyl, pyrrolyl, or pyrrolidinyl.

6. The compound of claim 1, which is
1-(3-(1-Imidazolyl)phenyl)-5-(methoxymethyl)benzimidazole;
1-(3-(1-Imidazolyl)phenyl)-5-(ethoxymethyl)benzimidazole;
1-(3-(1-Imidazolyl)phenyl)-5-(isopropoxymethyl)benzimidazole;
1-(3-(1-Imidazolyl)phenyl)-5-(propoxymethyl)benzimidazole;
1-(3-(1-Imidazolyl)phenyl)-5-(cyclopropylmethoxymethyl)benzimidazole;
1-(3-(1-Pyrazolyl)phenyl)-5-(methoxymethyl)benzimidazole;
1-(3-(1-Pyrazolyl)phenyl)-5-(hydroxymethyl)benzimidazole;
1-(3-(1-Pyrazolyl)phenyl)-5-(ethoxymethyl)benzimidazole;
1-(3-(1-Pyrazolyl)phenyl)-5-(isopropoxymethyl)benzimidazole;
1-(3-(1-Pyrazolyl)phenyl)-5-(propoxymethyl)benzimidazole;
1-(3-(1-Pyrazolyl)phenyl)-5-(cyclopropylmethoxymethyl)benzimidazole;
1-(3-(3-Furyl)phenyl)-5-(methoxymethyl)benzimidazole;
1-(3-(3-Furyl)phenyl)-5-(hydroxymethyl)benzimidazole;
1-(3-(3-Furyl)phenyl)-5-(ethoxymethyl)benzimidazole;
1-(3-(3-Furyl)phenyl)-5-(isopropoxymethyl)benzimidazole;
1-(3-(3-Furyl)phenyl)-5-(propoxymethyl)benzimidazole;
1-(3-(3-Furyl)phenyl)-5-(cyclopropylmethoxymethyl)benzimidazole;
1-(3-(2-Thienyl)phenyl)-5-(methoxymethyl)benzimidazole;
1-(3-(2-Thienyl)phenyl)-5-(hydroxymethyl)benzimidazole;
1-(3-(2-Thienyl)phenyl)-5-(ethoxymethyl)benzimidazole;
1-(3-(2-Thienyl)phenyl)-5-(isopropoxymethyl)benzimidazole;
1-(3-(2-Thienyl)phenyl)-5-(propoxymethyl)benzimidazole;
1-(3-(2-Thienyl)phenyl)-5-(cyclopropylmethoxymethyl)benzimidazole;
5-(1-Hydroxyethyl)-1-(3-(3-isoxazolyl)phenyl)benzimidazole;
5-(1-(Allyloxy)ethyl)-1-(3-(3-isoxazolyl)phenyl)benzimidazole;
5-(1-(Propargyloxy)ethyl)-1-(3-(3-isoxazolyl)phenyl)benzimidazole;
5-(1-Methoxyethyl)-1-(3-(3-isoxazolyl)phenyl)benzimidazole;
5-(1-Ethoxyethyl)-1-(3-(3-isoxazolyl)phenyl)benzimidazole;
5-(1-Propoxyethyl)-1-(3-(3-isoxazolyl)phenyl)benzimidazole;
5-(1-Isopropoxyethyl)-1-(3-(3-isoxazolyl)phenyl)benzimidazole;
5-(1-(Cyclopropylmethoxy)ethyl)-1-(3-(3-isoxazolyl)phenyl)benzimidazole;
5-(1-Hydroxyethyl)-1-(3-(1-imidazolyl)phenyl)benzimidazole;
5-(1-Methoxyethyl)-1-(3-(1-imidazolyl)phenyl)benzimidazole;
5-(1-Ethoxyethyl)-1-(3-(1-imidazolyl)phenyl)benzimidazole;
5-(1-Propoxyethyl)-1-(3-(1-imidazolyl)phenyl)benzimidazole;
5-(1-Isopropoxyethyl)-1-(3-(1-imidazolyl)phenyl)benzimidazole;
5-(1-(Cyclopropylmethoxy)ethyl)-1-(3-(1-imidazolyl)phenyl)benzimidazole;
5-(1-Hydroxyethyl)-1-(3-(1-pyrrolidinyl)phenyl)benzimidazole;
5-(1-Methoxyethyl)-1-(3-(1-pyrrolidinyl)phenyl)benzimidazole;
5-(1-Ethoxyethyl)-1-(3-(1-pyrrolidinyl)phenyl)benzimidazole;
5-(1-Propoxyethyl)-1-(3-(1-pyrrolidinyl)phenyl)benzimidazole;
5-(1-Isopropoxyethyl)-1-(3-(1-pyrrolidinyl)phenyl)benzimidazole;
5-(1-(Cyclopropylmethoxy)ethyl)-1-(3-(1-pyrrolidinyl)phenyl)benzimidazole;
5-(2-Hydroxy-2-propyl)-1-(3-(1-pyrrolidinyl)phenyl)benzimidazole;
5-(2-Methoxy-2-propyl)-1-(3-(1-pyrrolidinyl)phenyl)benzimidazole;
5-(2-Ethoxy-2-propyl)-1-(3-(1-pyrrolidinyl)phenyl)benzimidazole;
5-(2-Propoxy-2-propyl)-1-(3-(1-pyrrolidinyl)phenyl)benzimidazole;
5-(2-Isopropoxy-2-propyl)-1-(3-(1-pyrrolidinyl)phenyl)benzimidazole;
5-(2-(Cyclopropylmethoxy)-2-propyl)-1-(3-(1-pyrrolidinyl)phenyl)benzimidazole;
5-(Hydroxymethyl)-1-(3-(2-thiazolyl)phenyl)benzimidazole;
5-(Hydroxymethyl)-1-(3-(1-imidazolyl)phenyl)benzimidazole;
5-(Hydroxymethyl)-1-(3-(3-isoxazolyl)phenyl)benzimidazole;
5-(Hydroxymethyl)-1-(3-(1-pyrrolyl)phenyl)benzimidazole;
5-(Methoxymethyl)-1-(3-(2-thiazolyl)phenyl)benzimidazole;
5-(Methoxymethyl)-1-(3-(3-isoxazolyl)phenyl)benzimidazole;
5-(Ethoxymethyl)-1-(3-(3-isoxazolyl)phenyl)benzimidazole;
5-(Isopropoxymethyl)-1-(3-(3-isoxazolyl)phenyl)benzimidazole;
5-(Propoxymethyl)-1-(3-(3-isoxazolyl)phenyl)benzimidazole;
5-(Cyclopropylmethoxymethyl)-1-(3-(3-isoxazolyl)phenyl)benzimidazole;
5-(Methoxymethyl)-1-(3-(1-pyrrolyl)phenyl)benzimidazole;
5-(Ethoxymethyl)-1-(3-(1-pyrrolyl)phenyl)benzimidazole;
5-(Isopropoxymethyl)-1-(3-(1-pyrrolyl)phenyl)benzimidazole;
5-(Propoxymethyl)-1-(3-(1-pyrrolyl)phenyl)benzimidazole;
5-(Cyclopropylmethoxymethyl)-1-(3-(1-pyrrolyl)phenyl)benzimidazole;
5-(Ethoxymethyl)-1-(3-(2-thiazolyl)phenyl)benzimidazole;
5-(Isopropoxymethyl)-1-(3-(2-thiazolyl)phenyl)benzimidazole;
5-(Propoxymethyl)-1-(3-(2-thiazolyl)phenyl)benzimidazole;
5-(Cyclopropylmethoxymethyl)-1-(3-(2-thiazolyl)phenyl)benzimidazole;
or an N-oxide thereof, or any of its isomers or any mixture of its isomers,
or a pharmaceutically acceptable salt thereof.

7. A pharmaceutical composition, comprising a therapeutically effective amount of a compound of claim 1, or an N-oxide thereof, or any of its isomers or any mixture of its isomers, or a pharmaceutically acceptable salt thereof, together with at least one pharmaceutically acceptable carrier, excipient or diluent.

8. A method for treatment, prevention or alleviation of a disease or a disorder or a condition of a living animal body, including a human, which disorder, disease or condition is responsive to modulation of the GABA_{A} receptor complex in the central nervous system, which method comprises the step of administering to such a living animal body in need thereof a therapeutically effective amount of a compound according to claim 1, or an N-oxide thereof, or any of its isomers or any mixture of its isomers, or a pharmaceutically acceptable salt thereof.

9. The method according to claim 8, for the manufacture of a pharmaceutical composition for the treatment, prevention or alleviation of a disease or a disorder or a condition of a mammal, including a human, which disease, disorder or condition is responsive to modulation of the GABA_{A} receptor complex in the central nervous system.

10. The method according to claim 9, wherein the disease, disorder or condition is anxiety disorders, panic disorder with or without agoraphobia, agoraphobia without history of panic disorder, animal and other phobias, social phobias, obsessive-compulsive disorder, and generalized or substance-induced anxiety disorder; stress disorders, post-traumatic and acute stress disorder, sleep disorders, memory disorder, neuroses, convulsive disorders, epilepsy, febrile convulsions in children, migraine, depressive or bipolar disorders, single-episode or recurrent major depressive disorder, dysthymic disorder, bipolar I and bipolar II manic disorders, cyclothymic disorder, psychotic disorders, including schizophrenia, neurodegeneration arising from cerebral ischemia, attention deficit hyperactivity disorder, pain, nociception, emesis, acute, delayed and anticipatory emesis, particular emesis induced by chemotherapy or radiation, motion sickness, post-operative nausea, vomiting, eating disorders, anorexia nervosa, bulimia nervosa, premenstrual syndrome, muscle spasm, spasticity, e.g. in paraplegic patients, the effects of substance abuse or dependency, alcohol withdrawal, cognitive disorders, Alzheimer's disease, or disorders of circadian rhythm, e.g. in subjects suffering from the effects of jet lag or shift work.

11. (canceled)
